(19) United States
(12) Patent Application Publication
(10) Pub. No.: US 2026/0130768 A1
(43) Pub. Date: May 14, 2026

(54) TALAR IMPLANT

(71) Applicant: WRIGHT MEDICAL TECHNOLOGY, INC., Memphis, TN (US)

(72) Inventor: Meghan KUBACKI, Cookeville, TN (US)

(73) Assignee: WRIGHT MEDICAL TECHNOLOGY, INC., Memphis, TN (US)

(21) Appl. No.: 19/443,302

(22) Filed: Jan. 8, 2026

Related U.S. Application Data
(63) Continuation of application No. 18/001,942, now Pat. No. 12,539,218.
(60) Provisional application No. 63/106,403, filed on Oct. 28, 2020.

(51) Int. Cl. A61F 2/42; A61F 2/30

(52) U.S. Cl. CPC A61F 2/4202; A61F 2002/30057; A61F 2002/30383; A61F 2002/30405; A61F 2002/305; A61F 2002/30622; A61F 2002/4207

(57) ABSTRACT

Provided is a total talar replacement prosthesis that has a metallic body shell and a metallic or biologic core, where the metallic body shell includes one or both of a removable calcaneus attachment and a removable navicular attachment. The attachments, when removed, exposes a respective calcaneus-facing surface or a navicular-facing surface that can enhance fusion to the respective bones, calcaneus and/or navicular.

### CROSS-REFERENCE TO RELATED APPLICATION

This application is a continuation filed under 37 C.F.R. § 1.53 claiming the benefit under 35 U.S.C. § 120 of any and all applications for which a foreign or domestic priority claim is identified in the Application Data Sheet as filed with the present application, including U.S. patent application Ser. No. 18/001,942, filed Dec. 15, 2022, which is a national stage application, filed under 35 U.S.C. 371, of International Patent Application No. PCT/US 2021/071308, filed Aug. 30, 2021, which claims priority to U.S. Provisional Patent Application No. 63/106,403, filed on Oct. 28, 2020, and are hereby incorporated by reference in accordance with 37 C.F.R. §§ 1.57; 1.97; and 1.98 in their entireties.

### FIELD OF DISCLOSURE

Disclosed is a total talar replacement prosthesis.

### BACKGROUND

Talar necrosis or talar implant subsidence can leave little to no talar body with no options left for the patient than ankle fusion or amputation. As amputation is not a particularly desirable option, fusion might be preferred by many patients. However, currently available total talar implants do not allow for fusion with either the navicular and/or calcaneus. Therefore, there is a need for a better total talar implant that allow for such fusions.

### SUMMARY

Provided is a total talar replacement prosthesis embodiment that comprise a metallic body shell and a metallic or biologic core, where the metallic body shell comprises a removable calcaneus attachment that, when removed, exposes a calcaneus-facing surface. The calcaneus-facing surface can comprise a portion that is a discontinuity in the metallic body shell that exposes the metallic or biologic core that can enable the total talar replacement prosthesis to fuse to a calcaneus bone. In some embodiments, the core can be a solid metal core, a metal cancellous matrix core, a solid biologic core, or a biologic cancellous matrix core. The exposed metal or biologic core can be directly bonded to a calcaneus bone and the cancellous matrix structure can enhance bone ingrowth or on-growth to fuse the prosthesis to the calcaneus bone.

Also provided is a total talar replacement prosthesis embodiment that comprise a metallic body shell and a metallic or biologic core, where the metallic body shell comprises a removable navicular attachment that, when removed, exposes a navicular-facing surface. The navicular-facing surface can comprise a portion that is a discontinuity in the metallic body shell that exposes the metallic or biologic core that can enable the total talar replacement prosthesis to fuse to a navicular bone. In some embodiments, the core can be a solid metal core, a metal cancellous matrix core, a solid biologic core, or a biologic cancellous matric core. The exposed metal or biologic core can be directly bonded to a navicular bone and the cancellous matrix structure can enhance bone ingrowth or on-growth to fuse the prosthesis to the navicular bone.

Also provided is a total talar replacement prosthesis embodiment that comprises a metallic body shell that has both the removable navicular attachment and the removable calcaneus attachment.

### BRIEF DESCRIPTION OF THE DRAWINGS

FIG. 1A is an illustration of an embodiment of the total talar replacement prosthesis comprising a navicular-facing surface according to the present disclosure with its navicular attachment component removed.

FIG. 1B is an illustration of a navicular attachment component of the total talar replacement prosthesis according to the present disclosure.

FIG. 2A is an illustration of an embodiment of the total talar replacement prosthesis comprising a calcaneus-facing surface according to the present disclosure with its calcaneus attachment component removed.

FIG. 2B is an illustration of a calcaneus attachment component of the total talar replacement prosthesis according to the present disclosure.

FIG. 3 is an illustration of another embodiment of the total talar replacement prosthesis according to the present disclosure with both the navicular attachment component and the calcaneus attachment component removed.

FIG. 4 is an illustration of a total talar replacement prosthesis of FIG. 1A, FIG. 2A, or FIG. 3, with their navicular attachment and/or calcaneus attachment components in place.

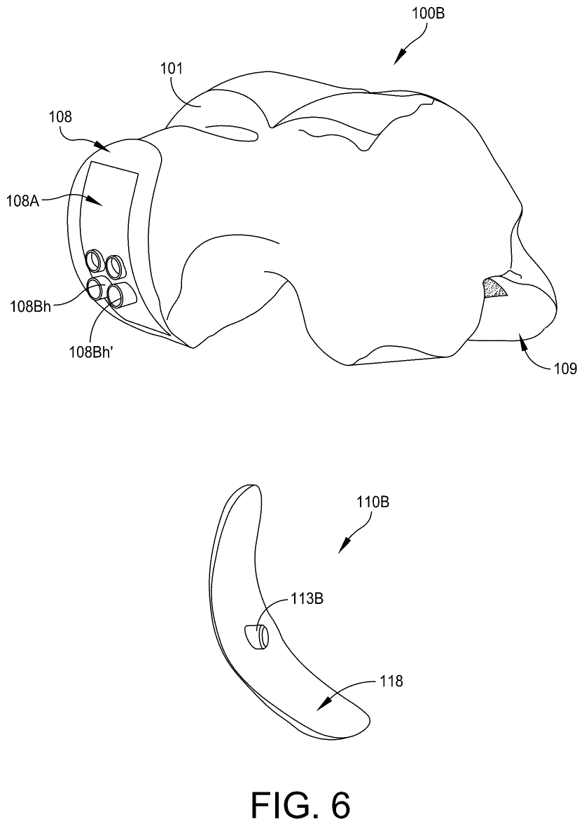

FIGS. 5-7 are illustrations of additional embodiments of the total talar replacement prosthesis according to the present disclosure.

FIG. 8 is a flowchart of an example of a method for installing the total talar replacement prosthesis of the present disclosure.

### DETAILED DESCRIPTION

This description of the exemplary embodiments is intended to be read in connection with the accompanying drawings, which are to be considered part of the entire written description. The drawing figures are not necessarily to scale, and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

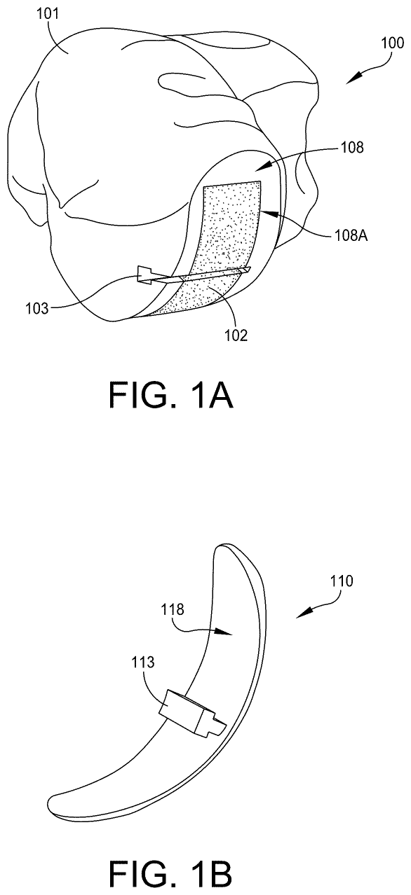

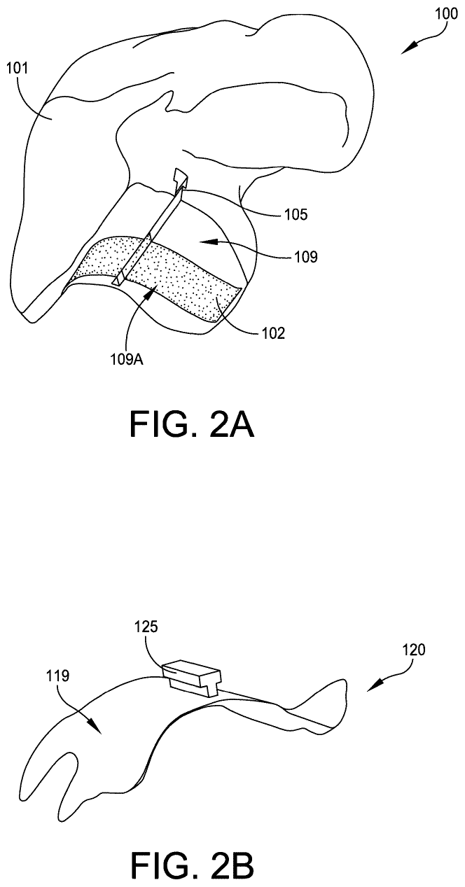

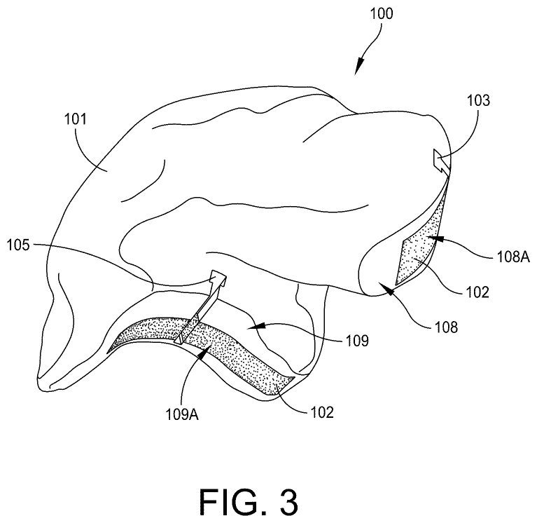

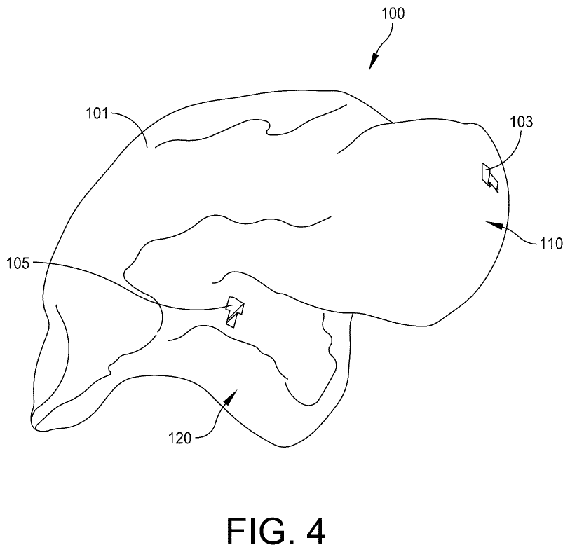

Referring to FIGS. 1A and 2A, provided is a total talar replacement prosthesis 100 comprising a metallic body shell 101 and a core 102. The metallic body shell 101 comprises a removable calcaneus attachment 120 that, when removed, exposes a calcaneus-facing surface 109. This feature can be useful when the condition of the patient's ankle is such that fusing the talar replacement prosthesis 100 and the calcaneus is desired. By removing the calcaneus attachment 120 and contacting the calcaneus-facing surface 109 directly against the calcaneus, the appropriate core material can facilitate the talar replacement prosthesis 100 to fuse to the calcaneus. The calcaneus-facing surface 109 comprises a core exposing portion 109A that is a discontinuity in the metallic body shell 101 that exposes the core 102.

Referring to FIG. 2B, the calcaneus attachment 120 is configured to be removably attached to the calcaneus-facing surface 109 and cover at least a portion of the exposed core 102 in the exposed core portion 109A. In some embodiments, the calcaneus attachment 120 is sufficiently large to completely cover the exposed core portion 109A.

To removably secure the calcaneus attachment 120 to the calcaneus-facing surface 109, in some embodiments, the calcaneus-facing surface 109 comprises a first attachment feature and the calcaneus attachment 120 comprises a corresponding mating attachment feature 125. In the illustrated example shown in FIG. 2B, the first attachment feature is a groove 105 and the mating attachment feature is a rail portion 125 that is configured to engage the groove on the calcaneus-facing surface by sliding. In some embodiments, the first attachment feature and the mating attachment feature are configured to engage each other by threading or clipping/snapping.

As shown in FIGS. 1A and 1B, the metallic body shell 101 of the total talar replacement prosthesis 100 also comprises a removable navicular attachment 110 that, when removed, exposes a navicular-facing surface 108. This feature can be useful when the condition of the patient's ankle is such that fusing the talar replacement prosthesis 100 and the navicular is desired. By removing the navicular attachment 110 and contacting the navicular-facing surface 108 directly against the navicular, the appropriate core material can facilitate the talar replacement prosthesis 100 to fuse to the navicular. The navicular-facing surface 108 comprises a core exposing portion 108A that is a discontinuity in metallic body shell 101 that exposes the core 102.

Referring to FIG. 1B, the navicular attachment 110 is configured to be removably attached to the navicular-facing surface 108 and cover at least a portion of the exposed core 102 in the exposed core portion 108A. In some embodiments, the navicular attachment 110 is sufficiently large to completely cover the exposed core portion 108A.

To removably secure the navicular attachment 110 to the navicular-facing surface 108, in some embodiments, the navicular-facing surface 108 comprises a first attachment feature and the navicular attachment 110 comprises a corresponding mating attachment feature 113. In the illustrated example shown in FIG. 1B, the first attachment feature is a groove 103 and the mating attachment feature is a rail portion 113 that is configured to engage the groove on the navicular-facing surface by sliding.

In some embodiments, the first attachment feature and the mating attachment feature are configured to engage each other by threading or clipping/snapping. Referring to FIGS. 6 and 7, examples of embodiments of total talar replacement prosthesis utilizing such first attachment feature and corresponding mating attachment feature are disclosed. In the examples shown in FIGS. 6 and 7, a navicular-facing surface and navicular attachment examples are used to illustrate such attachment configurations. FIG. 6 shows a total talar replacement prosthesis 100B comprising a navicular facing surface 108 comprising an exposed core portion 108A and a corresponding navicular attachment 110B that can be removably attached to the exposed core portion 108A. The removable attachment is achieved by a snap-fitting attachment feature. Provided in the exposed core portion 108A are a plurality of cylindrical structures 108Bh as the first attachment feature. Provided in the navicular attachment 110B is at least one stud 113B that is the mating attachment feature configured to snap-fit into the first attachment feature, the plurality of cylindrical structures 108Bh. When the navicular attachment 110B is properly aligned with the exposed core portion 108A, the at least one stud 113B snap-fits in the geometric center of the plurality of cylindrical structures 108Bh. Each of the plurality of cylindrical structures 108Bh can also comprise a ridge 108Bh′ provided along the perimeter of the cylindrical structure for enabling the snap-fitting engagement. FIG. 7 shows a total talar replacement prosthesis 100C comprising a navicular-facing surface 108 comprising an exposed core portion 108A and a corresponding navicular attachment 110C that can be removably attached to the exposed core portion 108A. Provided in the exposed core portion 108A is at least one hole 108Ch as the first attachment feature. Provided in the navicular attachment 110C is at least one pin 113C that is the mating attachment feature configured to snap-fit into the at least one hole 108Ch, first attachment feature. In the example shown, the at least one hole 108Ch is configured with a retaining ring 108 ring and the at least one pin 113C is configured with a beveled leading edge 113CB and an annular groove 113CR at a set distance from the beveled leading edge 113CB. The retaining ring 118 ring has an inner diameter that is smaller than the outer diameter of the at least one pin 113C. When the at least one pin 113C is inserted into the at least one hole 108Ch, the beveled leading edge 113CB elastically expands the retaining ring 108 ring and advances into the at least one hole 108Ch. Once the at least one pin 113C advances sufficient depth into the hole 108Ch, the annular groove 113CR reaches the retaining ring 108 ring and the retaining ring 108 ring contracts back into its initial diameter and engages the annular groove 113CR, thus retaining the navicular attachment 110C in place.

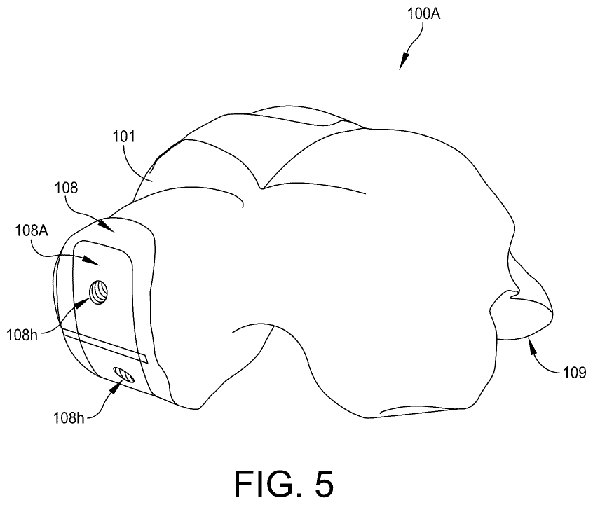

Referring to FIG. 3, in some embodiments, the total talar replacement prosthesis 100 can be configured to have both the navicular-facing surface 108 and the calcaneus-facing surface 109 and the corresponding navicular attachment 110 and the calcaneus attachment 120. Such embodiment can be used in situations where fusing the talar replacement prosthesis 100 to both the calcaneus and navicular is desired. The various options for enhancing or facilitating the fusion between the talar replacement prosthesis 100 and the calcaneus and the navicular described above are also applicable to this embodiment.

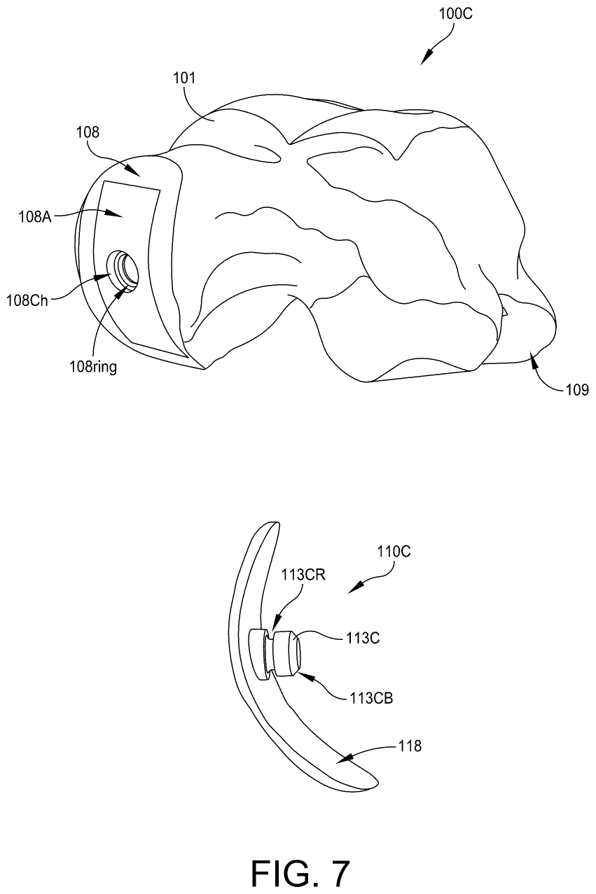

According to an aspect of the present disclosure, the core 102 can be a solid metal core where the core is of the same metal as the metallic body shell 101 or the core is of a different material as the metallic body shell 101, such as a resorbable metal, a soft metal, or a biologic material. In some embodiments, the core 102 can have a cancellous matrix formed of a porous metallic material such as Wright Medical Technology's ADAPTIS™. or of a porous biologic material such as Wright Medical Technology's ALLOPURE™. Whether the core 102 is a solid core or a cancellous matrix core, the exposed core portions 108A and/or 109A can be provided with threaded features imbedded therein to allow compression for fusion with their respective navicular bone or calcaneus bone. FIG. 5 shows an example of a total talar replacement prosthesis 100 in which the exposed core portion 108A on the navicular-facing surface 108 is provided with at least one threaded blind hole 108h. The at least one threaded blind hole 108h would allow insertion of at least one bone screw through the navicular and/or calcaneus bone and into the total talar replacement prosthesis.

In embodiments where the core 102 is a cancellous matrix core, the exposed core portions 108A and/or 109A can be directly bonded to a navicular and/or calcaneus bone, respectively, and the cancellous matrix structure can enhance bone ingrowth or on-growth and help fuse the prosthesis to the navicular and/or calcaneus bone.

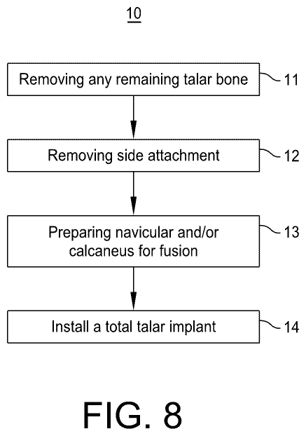

Referring to the flowchart 10 in FIG. 8, an example of a method for implanting the total talar replacement prosthesis 100 of the present disclosure is disclosed. The method can include removing any remaining talar bone from the patient's ankle (step 11); removing side attachment for either a talo-navicular fusion and/or talo-calcaneus fusion (step 12); preparing navicular and/or calcaneus for fusion (step 13); and installing the total talar implant 100 (step 14) of the present disclosure.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

What is claimed is:

1. A total talar replacement prosthesis comprising:
a metallic shell that encloses a core, wherein the metallic shell includes a navicular-facing surface that defines a discontinuity sized and arranged so as to expose a portion of the core; and
a navicular attachment configured to be removably attached to the navicular-facing surface so as to be positioned relative to the discontinuity to thereby cover the exposed portion of the core by at least partially filling the discontinuity, wherein the navicular-facing surface of the metallic shell further defines a plurality of recesses each having a ridge along a perimeter and the navicular attachment includes at least one stud that is sized and positioned so as to engage a correspondingly positioned and sized one of the plurality of recesses such that the at least one stud forms a snap-fit by engagement with the ridge at the perimeter of the correspondingly positioned and sized one of the plurality of recesses.

2. The total talar replacement prosthesis of claim 1, wherein the core is at least one of a metallic and a biologic cancellous matrix, and the exposed portion of the cancellous matrix on the navicular-facing surface is configured to enhance at least one of bone ingrowth and on-growth to fuse the total talar replacement prosthesis prosthesis to a navicular bone.

3. The total talar replacement prosthesis of claim 1, wherein the navicular attachment is sufficiently large to completely cover the exposed portion of the core at the navicular-facing surface.

4. The total talar replacement prosthesis of claim 1, wherein the metallic shell further includes a calcaneus-facing surface defining a discontinuity that is sized and arranged so as to expose a portion of the core, and a calcaneus attachment configured to be removably attached to the calcaneus-facing surface and to removably cover the exposed portion of the core by at least partially filling the discontinuity.

5. The total talar replacement prosthesis of claim 4, wherein the calcaneus-facing surface includes, within its discontinuity, a plurality of recesses each having a ridge along a perimeter and the calcaneus attachment includes at least one stud that is sized and positioned so as to engage a correspondingly positioned and sized one of the plurality of recesses such that the at least one stud forms a snap-fit by engagement with the ridge at the perimeter of the correspondingly positioned and sized one of the plurality of recesses.

6. The total talar replacement prosthesis of claim 1, wherein comprises a groove on the navicular-facing surface arranged in matting relationship with a rail disposed on the navicular attachment that is configured to engage the groove by sliding.

7. The total talar replacement prosthesis of claim 4, wherein the plurality of recesses comprises a groove on the calcaneus-facing surface arranged in matting relationship with a rail disposed on the calcaneus attachment that is configured to engage the groove by sliding.

8. The total talar replacement prosthesis of claim 1, wherein the core comprises at least one of a solid metal and a solid biologic.

9. The total talar replacement prosthesis of claim 8, wherein the material forming the core is different from the metallic shell and comprises at least one of a resorbable metal, a soft metal, and a biologic material.

10. The total talar replacement prosthesis of claim 2, wherein the biologic cancellous matrix includes at least one of a porous metallic material and a porous biologic material selected to facilitate bone bonding at the exposed portion of the core.

11. A total talar replacement prosthesis comprising:
a shell that partially encloses a core, wherein the shell includes a navicular-facing surface defining a discontinuity that exposes a first portion of the core and a calcaneus-facing surface defining a discontinuity that exposes a second portion of the core;
a navicular attachment configured to be removably attached to the navicular-facing surface and to removably cover at least a portion of the exposed first portion of the core by at least partially filling the discontinuity; and
a calcaneus attachment configured to be removably attached to the calcaneus-facing surface and to removably cover at least a portion of the exposed second portion of the core by at least partially filling the discontinuity, wherein at least one of the first and second portions includes at least one threaded blind hole oriented to receive a bone screw advanced into the exposed core to generate compression across a fusion interface.

12. The total talar replacement prosthesis of claim 11, wherein the threaded blind hole is provided in the first portion of the core at the navicular-facing surface and is configured to receive a screw advanced through the navicular bone into the core to allow compression for fusion with the navicular.

13. The total talar replacement prosthesis of claim 11, wherein the threaded blind hole is provided in the second portion of the core at the calcaneus-facing surface and is configured to receive a screw advanced through the calcaneus bone into the second portion to allow compression for fusion with the calcaneus.

14. The total talar replacement prosthesis of claim 11, wherein the core is solid.

15. The total talar replacement prosthesis of claim 11, wherein the core is at least one of a metallic and a biologic cancellous matrix and the threads are embedded within an exposed cancellous matrix to permit compression for fusion.

16. The total talar replacement prosthesis of claim 11, wherein both the navicular-facing surface and the calcaneus-facing surface include respective threaded blind holes positioned to receive screws advanced from both bones to generate bilateral compression across respective fusion interfaces.

17. The total talar replacement prosthesis of claim 11, wherein the navicular attachment and the calcaneus attachment are removable to select unilateral or bilateral fusion, and after removal the exposed first and second portions of the core are positioned to directly contact the navicular and/or calcaneus to promote fusion.

18. The total talar replacement prosthesis of claim 11, wherein the core is formed from a cancellous matrix so as to be directly bondable to bone and configured to enhance at least one of bone ingrowth and on-growth at the first and second exposed core portions.

19. The total talar replacement prosthesis of claim 11, wherein at least one of the navicular attachment and the calcaneus attachment is sufficiently large to completely cover its respective exposed first and second portions when installed.

20. The total talar replacement prosthesis of claim 11, wherein a groove is defined on the navicular-facing surface and the navicular attachment includes a rail configured and positioned so as to slide into the groove.

21. The total talar replacement prosthesis of claim 11, wherein a groove is defined on the calcaneus-facing surface and calcaneus attachment includes a rail configured and positioned so as to slide into the groove.

22. The total talar replacement prosthesis of claim 11, wherein the core comprises a material different from the shell, selected from the group consisting of a resorbable metal, a soft metal, and a biologic.

23. A method of implanting a total talar replacement prosthesis, the method comprising:
removing any remaining talar bone from a patient's ankle;
removing a side attachment corresponding to either a talo-navicular fusion or a talo-calcaneus fusion to expose a discontinuity that exposes a portion of the core at at least one of a navicular-facing surface and a calcaneus-facing surface;
preparing the corresponding bone surface for fusion;
positioning the total talar replacement prosthesis so that the exposed core portion directly contacts at least one of the navicular and calcaneus; and
advancing a bone screw from at least one of the navicular and calcaneus into at least one threaded blind hole provided in the exposed portion of the core to thereby generate compression across a fusion interface.

24. The method of claim 23, wherein the threaded blind hole is provided in the exposed core portion of the navicular-facing surface and the screw is advanced through the navicular into the threaded blind hole to compress the navicular-facing interface.

25. The method of claim 23, wherein the threaded blind hole is provided in the exposed core portion of the calcaneus-facing surface and the screw is advanced through the calcaneus into the threaded blind hole to compress the calcaneus-facing interface.

26. The method of claim 23, further comprising selecting bilateral fusion by removing both the navicular attachment and the calcaneus attachment and advancing screws from both the navicular and the calcaneus into respective threaded blind holes provided in the exposed core portions.

27. The method of claim 23, wherein the core comprises at least one of a metallic and biologic cancellous matrix core, and positioning the total talar replacement prosthesis includes orienting the exposed cancellous matrix core for direct bonding and enhanced bone ingrowth or on-growth at the bone-contacting surface.

28. The method of claim 23, wherein the total talar replacement prosthesis further comprises a groove-and-rail engagement between the metallic body shell and each removable attachment, and removing the side attachment includes sliding a rail of the attachment out of a groove of the corresponding facing surface to expose the discontinuity.
